(19) United States
(12) Patent Application Publication
(10) Pub. No.: US 2026/0228952 A1
(43) Pub. Date: Aug. 6, 2026

(54) MULTI-DIRECTION DIFFUSION WEIGHTED IMAGING ON PORTABLE, LOW-FIELD MAGNETIC RESONANCE IMAGING

(71) Applicant: Hyperfine Operations, Inc., Guilford, CT (US)

(72) Inventor: Rafael O'Halloran, Guilford, CT (US)

(73) Assignee: Hyperfine Operations, Inc., Guilford, CT (US)

(21) Appl. No.: 19/636,708

(22) Filed: Apr. 1, 2026

Related U.S. Application Data
(63) Continuation of application No. PCT/US2024/049664, filed on Oct. 2, 2024.
(60) Provisional application No. 63/587,277, filed on Oct. 2, 2023.

(51) Int. Cl. G06T 12/20; G16H 30/20

(52) U.S. Cl. CPC G06T 12/20; G16H 30/20

(57) ABSTRACT

Systems and methods for low-field multi-directional diffusion-weighted magnetic resonance imaging process are provided. One or more output images are generated based on multi-directional image reconstructions. Motion may be quantified based on signal variation. Certain data may be rejected based on the quantified motion. Image reconstructions may exclude data based on the quantified motion.

### CROSS-REFERENCE TO RELATED APPLICATIONS

This application is a bypass continuation of International Patent Application PCT/US2024/049664 filed October 2, 2024, which claims priority to U.S. Provisional Patent Application No. 63/587,277 filed October 2, 2023, the contents of each of which are incorporated herein by reference.

### BACKGROUND

Magnet resonance imaging (MRI) systems may be utilized to generate images of the inside of the human body. MRI systems may be used to detect magnetic resonance (MR) signals in response to applied electromagnetic fields. MRI techniques may include diffusion weighted imaging (DWI), via which water diffusion is used to investigate the white matter activity in the human brain. DWI processing techniques may be utilized with the goal of addressing resolution issues for images produced using DWI. Multi-shot images complicate image reconstruction because changing patient motion across different shots causes phase inconsistency.

### SUMMARY

At least one aspect of the present disclosure is directed to a low-field multi-directional diffusion-weighted magnetic resonance imaging process, the method comprising: quantifying motion based on signal variation; and generating one or more output images based on multi-directional image reconstructions that exclude data based on the quantified motion.

In various embodiments, the method comprises acquiring imaging data. In various embodiments, the imaging data is acquired using an MRI system with a field strength no greater than 0.2 T. In various embodiments, the MRI system is a portable MRI system that is cartable between locations using one or more battery-powered motors coupled to a set of wheels. In various embodiments, acquiring imaging data comprises performing a pseudo-trace acquisition encoded along a plurality of axes. In various embodiments, the multi-directional image reconstructions correspond to a first direction and a second direction that is different from the first direction. In various embodiments, the first direction is orthogonal to the second direction. In various embodiments, the multi-directional image reconstructions correspond to three different directions. In various embodiments, the method comprises aligning navigator signal across all segments. In various embodiments, the method comprises generating a reference pattern from an aligned navigator signal for motion level quantification. In various embodiments, the method comprises computing normalized projection residual against a reference pattern. In various embodiments, the method comprises determining for each segment, based on a projection having a normalized residual energy level above a chosen threshold, to reject data from the segment. In various embodiments, it may be determined to exclude all of the data based on the quantified motion, or only exclude a portion of the data based on the quantified motion. In various embodiments, it may be that no data is excluded based on the quantified motion.

At least another aspect of the present disclosure is directed to a low-field MRI system comprising one or more processors configured to perform any method disclosed herein. In various embodiments, the MRI system has a field strength no greater than 0.2 T. In various embodiments, the MRI system is cartable between locations using one or more battery-powered motors coupled to a set of wheels.

At least another aspect of the present disclosure is directed to a method of magnetic resonance imaging (MRI), the method comprising: performing, using an MRI system, a multi-shot, diffusion-weighted acquisition with diffusion-encoding along a plurality of physical axes to obtain imaging data of a subject in an imaging region of the MRI system, the imaging data comprising a plurality of shot segments; performing navigator-based motion adjustment, wherein performing the navigator-based motion adjustment comprises at least one of: aligning navigator signal across the shot segments in the multi-shot acquisition to obtain an aligned navigator signal; quantifying motion in the multi-shot acquisition; and/or rejecting data from shot segments based at least in part on the quantified motion; independently reconstructing a diffusion-weighted image corresponding to each of the plurality of axes to obtain a plurality of reconstructed images; and providing one or more output images based on the independently-reconstructed diffusion-weighted images.

In various embodiments, quantifying motion comprises computing normalized projection residual against a reference pattern. In various embodiments, the one or more output images are provided to at least one of (i) a display device or a computing device for presentation of the one or more output images, (ii) a non-transitory non-volatile computer-readable storage medium for storage of the one or more output images, and/or (iii) a computing system for subsequent accessibility of the one or more output images via a network. In various embodiments, the MRI system is a low-field MRI system. In various embodiments, the MRI system is a portable MRI system. In various embodiments, the one or more output images are based at least in part on (i) a first image without diffusion weighting and (ii) the independently-reconstructed diffusion-weighted images. In various embodiments, the first image is a b=0 s²/mm image and the diffusion-weighted images are b>100 s²/mm images. In various embodiments, generating the one or more output images comprises producing an apparent diffusion coefficient (ADC) map based at least in part on the plurality of reconstructed images. In various embodiments, the ADC map is based at least in part on a pseudo-trace obtained using the plurality of reconstructed images. In various embodiments, the pseudo-trace is based at least in part on an average of the plurality of reconstructed images. In various embodiments, the ADC map is produced using a pseudo-trace and an image without diffusion-weighting. In various embodiments, the one or more output images do not use rejected data.

At least one other aspect of the present disclosure is directed to a low-field multi-directional diffusion-weighted magnetic resonance imaging process, the method comprising quantifying motion based on signal variation, rejecting data based on the quantified motion, and generating one or more output images based on multi-directional image reconstructions that exclude rejected data.

In various embodiments, the method comprises acquiring imaging data by performing a pseudo-trace acquisition encoded along a plurality of physical axes. In various embodiments, the method comprises aligning a phase of a navigator signal across all segments. In various embodiments, the method comprises generating a reference pattern from an aligned navigator signal for motion level quantification. In various embodiments, the method comprises computing normalized projection residual against a reference pattern. In various embodiments, the method comprises determining for each segment, based on a projection having a normalized residual energy level above a chosen threshold, to reject data from the segment.

At least one other aspect of the present disclosure is directed to an MRI system comprising one or more processors and being configured to perform any of the methods disclosed herein.

In various embodiments, the MRI system is a low-field MRI system that has a field strength no greater than 0.2 T. In various embodiments, the MRI system is a portable MRI system that is cartable between locations using one or more battery-powered motors coupled to a set of wheels.

At least one other aspect of the present disclosure is directed to a non-transitory computer-readable medium comprising instructions executable by one or more processors to perform any of the methods disclosed above or any of the methods disclosed below.

In various embodiments, the one or more processors are part of any combination of an MRI system, a computing device, and/or a computing system.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects may be combined and it will be readily appreciated that features described in the context of one aspect of the present disclosure may be combined with other aspects. Aspects may be implemented in any convenient form. In a non-limiting example, by appropriate computer programs, which may be carried on appropriate carrier media (computer readable media), which may be tangible carrier media (e.g. disks) or intangible carrier media (e.g. communications signals). Aspects may also be implemented using suitable apparatus, which may take the form of programmable computers running computer programs arranged to implement the aspect. As used in the specification and in the claims, the singular form of “a,” “an,” and “the” include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing.

FIG. 1 illustrates example components of a magnetic resonance imaging system, in accordance with one or more implementations.

FIG. 2 illustrates an example system for estimating navigator maps for diffusion-weighted imaging (DWI) image reconstruction, in accordance with one or more implementations.

FIG. 3 provides a flowchart of example steps of imaging a subject in accordance with example embodiments of the disclosure.

FIG. 4 depicts an example method of estimating a navigator map for multi-shot MR data using a reference image and performing multi-shot MR image reconstruction using the navigator map, in accordance with one or more implementations.

FIG. 5 depicts a flowchart of an example method of rejecting imaging data, in accordance with one or more implementations.

FIG. 6 shows an example DWI image of a patient without motion correction, in accordance with one or more implementations.

FIG. 7 shows an example DWI image of a patient on which image correction has been performed, in accordance with one or more implementations.

FIG. 8 is a block diagram of an example computing system suitable for use in the various arrangements described herein, in accordance with one or more example implementations.

### DETAILED DESCRIPTION

Below are detailed descriptions of various concepts related to and implementations of techniques, approaches, methods, apparatuses, and systems for phase-navigated multi-shot MR image reconstruction using estimated convolution kernels. The various concepts introduced above and discussed in detail below may be implemented in any of numerous ways, as the described concepts are not limited to any particular manner of implementation. Examples of specific implementations and applications are provided primarily for illustrative purposes. Although illustrative examples as applied to DWI imaging are presented, the disclosed approaches are applicable to other multi-shot MR imaging modalities.

DWI imaging techniques may be utilized to image microscopic diffusivity, which may be particularly useful for investigating the white matter of the brain and performing a clinical assessment of diffusion within soft tissue. DWI imaging techniques utilize a combination of radiofrequency (RF) pulses and gradient magnetic fields to purposely wind and then rewind transverse magnetic spin phases, which results in each spin accruing a motion dependent phase. Incoherent motion, such as diffusion, manifests as spin phase incoherence, which induces cancellation of individual spin fields, and, ultimately, a decreased (e.g., diffusion weighted) electromagnetic signal level. The diffusion-caused signal loss may be viewed as an absolute image magnitude attenuation (scaling). This mechanism enables clinical assessment of diffusion, and is useful for various diagnostic processes.

Although DWI data may be acquired with single-shot pulse sequences, such as single-shot echo-planar imaging (EPI), such single-shot DWI techniques are limited in spatial resolution, which introduces challenges when measuring detailed diffusion properties in fine structures where high spatial resolution is required. To address these limitations, multi-shot techniques may be utilized to address the amplified shot-to-shot motion-induced phase variations and produce adequate high-resolution DWI data. Multi-shot DWI acquisition techniques involve multiple spatially encoded acquisitions, as opposed to single-shot, where an entire two-dimensional (2D) or 3D image would be encoded and acquired with a single excitation and single train of acquisitions.

All motion, regardless of its source and spin coherency, contributes to the phase accrual during DWI imaging. Coherent spin motion from various sources (e.g., patient movements such as respiration, convulsion, etc.), accumulates coherent spin phase, which differentiates it from diffusion and does not induce signal cancellation. The coherent phase therefore remains at the time of signal acquisition after the diffusion encoding module and exhibits as extra phase components on complex MR images (e.g., a motion phase map). Such phase maps are motion-dependent and may cause data inconsistency for certain types of acquisitions.

Clinical DWI images may be noisy. For diffusion sensitivity, MR imaging systems may utilize a high b-value (the metric of diffusion weighting, which quantitatively describes the amount of spin “winding” and “rewinding”) that substantially attenuates the signal. Three-dimensional (3D) imaging may also be utilized to boost DWI signal-to-noise-ratio (SNR), which typically requires multi-shot acquisition. Multi-shot acquisition techniques complicate DWI image reconstruction processes because changing patient motion across different shots causes phase inconsistency. Phase-navigation may be utilized to handle such phase inconsistency at image reconstruction, enabling successful reconstruction of the shot-invariant diffusion weighted image.

Model-based phase-navigated DWI reconstruction utilizes explicit knowledge of motion phase maps at each shot to establish the Fourier transform (FT) linear mapping between acquired k-space data of each shot and the diffusion-weighted image that may be modulated by the shot specific motion phase maps. The phase maps are generally estimated using partial or entire data of each shot (e.g., navigator data), particularly for in vivo scanning.

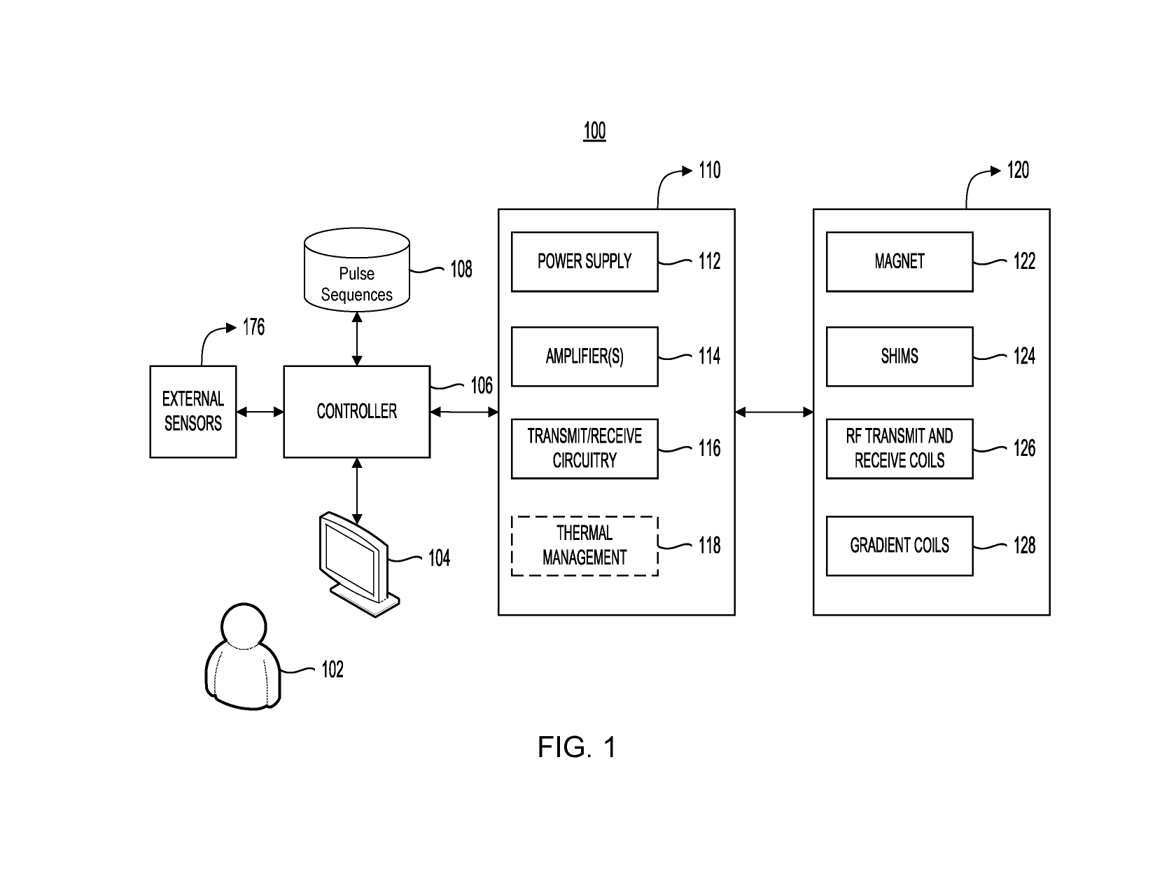

FIG. 1A illustrates an example MRI system which may be utilized in connection with the navigator map estimation techniques described herein. In FIG. 1A, MRI system 100 may include a computing device 104, a controller 106, a pulse sequences repository 108, a power management system 110, and magnetics components 120. The MRI system 100 is illustrative, and an MRI system may have one or more other components of any suitable type in addition to or instead of the components illustrated in FIG. 1A. Additionally, the implementation of components for a particular MRI system may differ from those described herein. Examples of low-field MRI systems may include portable MRI systems, which may have a field strength that may be, in a non-limiting example, less than or equal to 0.5 T, that may be less than or equal to 0.2 T, that may be within a range from 1 mT to 100 mT, that may be within a range from 50 mT to 0.1 T, that may be within a range of 40 mT to 80 mT, that may be about 64 mT, etc.

The magnetics components 120 may include B₀ magnets 122, shims 124, radio frequency (RF) transmit and receive coils 126, and gradient coils 128. The B₀ magnets 122 may be used to generate a main magnetic field B₀. B₀ magnets 122 may be any suitable type or combination of magnetics components that may generate a desired main magnetic B₀ field. In some embodiments, B₀ magnets 122 may be one or more permanent magnets, one or more electromagnets, one or more superconducting magnets, or a hybrid magnet comprising one or more permanent magnets and one or more electromagnets or one or more superconducting magnets. In some embodiments, B₀ magnets 122 may be configured to generate a B₀ magnetic field having a field strength that may be less than or equal to 0.2 T or within a range from 50 mT to 0.1 T.

In some implementations, the B₀ magnets 122 may include a first and second B₀ magnet, which may each include permanent magnet blocks arranged in concentric rings about a common center. The first and second B₀ magnet may be arranged in a bi-planar configuration such that the imaging region is located between the first and second B₀ magnets. In some embodiments, the first and second B₀ magnets may each be coupled to and supported by a ferromagnetic yoke configured to capture and direct magnetic flux from the first and second B₀ magnets.

The gradient coils 128 may be arranged to provide gradient fields and, in a non-limiting example, may be arranged to generate gradients in the B₀ field in three substantially orthogonal directions (X, Y, and Z). Gradient coils 128 may be configured to encode emitted MR signals by systematically varying the B₀ field (the B₀ field generated by the B₀ magnets 122 or shims 124) to encode the spatial location of received MR signals as a function of frequency or phase. In a non-limiting example, the gradient coils 128 may be configured to vary frequency or phase as a linear function of spatial location along a particular direction, although more complex spatial encoding profiles may also be provided by using nonlinear gradient coils. In some embodiments, the gradient coils 128 may be implemented using laminate panels (e.g., printed circuit boards), in a non-limiting example.

MRI scans are performed by exciting and detecting emitted MR signals using transmit and receive coils, respectively (referred to herein as radio frequency (RF) coils). The transmit and receive coils may include separate coils for transmitting and receiving, multiple coils for transmitting or receiving, or the same coils for transmitting and receiving. Thus, a transmit/receive component may include one or more coils for transmitting, one or more coils for receiving, or one or more coils for transmitting and receiving. The transmit/receive coils may be referred to as Tx/Rx or Tx/Rx coils to generically refer to the various configurations for transmit and receive magnetics components of an MRI system. These terms are used interchangeably herein. In FIG. 1A, RF transmit and receive coils 126 may include one or more transmit coils that may be used to generate RF pulses to induce an oscillating magnetic field B₁. The transmit coil(s) may be configured to generate any type of suitable RF pulses.

The power management system 110 includes electronics to provide operating power to one or more components of the MRI system 100. In a non-limiting example, the power management system 110 may include one or more power supplies, energy storage devices, gradient power components, transmit coil components, or any other suitable power electronics needed to provide suitable operating power to energize and operate components of MRI system 100. As illustrated in FIG. 1A, the power management system 110 may include a power supply system 112, power component(s) 114, transmit/receive circuitry 116, and may optionally include thermal management components 118 (e.g., cryogenic cooling equipment for superconducting magnets, water cooling equipment for electromagnets).

The power supply system 112 may include electronics that provide operating power to magnetic components 120 of the MRI system 100. The electronics of the power supply system 112 may provide, in a non-limiting example, operating power to one or more gradient coils (e.g., gradient coils 128) to generate one or more gradient magnetic fields to provide spatial encoding of the MR signals. Additionally, the electronics of the power supply system 112 may provide operating power to one or more RF coils (e.g., RF transmit and receive coils 126) to generate or receive one or more RF signals from the subject. In a non-limiting example, the power supply system 112 may include a power supply configured to provide power from mains electricity to the MRI system or an energy storage device. The power supply may, in some embodiments, be an AC-to-DC power supply that converts AC power from mains electricity into DC power for use by the MRI system. The energy storage device may, in some embodiments, be any one of a battery, a capacitor, an ultracapacitor, a flywheel, or any other suitable energy storage apparatus that may bi-directionally receive (e.g., store) power from mains electricity and supply power to the MRI system. Additionally, the power supply system 112 may include additional power electronics including, but not limited to, power converters, switches, buses, drivers, and any other suitable electronics for supplying the MRI system with power.

The amplifiers(s) 114 may include one or more RF receive (Rx) pre-amplifiers that amplify MR signals detected by one or more RF receive coils (e.g., coils 126), one or more RF transmit (Tx) power components configured to provide power to one or more RF transmit coils (e.g., coils 126), one or more gradient power components configured to provide power to one or more gradient coils (e.g., gradient coils 128), and may provide power to one or more shim power components configured to provide power to one or more shims (e.g., shims 124). In some implementations, the shims 124 may be implemented using permanent magnets, electromagnetics (e.g., a coil), or combinations thereof. The transmit/receive circuitry 116 may be used to select whether RF transmit coils or RF receive coils are being operated.

As illustrated in FIG. 1A, the MRI system 100 may include the controller 106 (also referred to as a console), which may include control electronics to send instructions to and receive information from power management system 110. The controller 106 may be configured to implement one or more pulse sequences, which are used to determine the instructions sent to power management system 110 to operate the magnetic components 120 in a desired sequence (e.g., parameters for operating the RF transmit and receive coils 126, parameters for operating gradient coils 128, etc.). Additionally, the controller 106 may execute processes to estimate navigator maps for DWI reconstruction according to various techniques described herein. A pulse sequence may generally describe the order and timing in which the RF transmit and receive coils 126 and the gradient coils 128 operate to acquire resulting MR data. In a non-limiting example, a pulse sequence may indicate an order and duration of transmit pulses, gradient pulses, and acquisition times during which the receive coils acquire MR data.

A pulse sequence may be organized into a series of periods. In a non-limiting example, a pulse sequence may include a pre-programmed number of pulse repetition periods, and applying a pulse sequence may include operating the MRI system in accordance with parameters of the pulse sequence for the pre-programmed number of pulse repetition periods. In each period, the pulse sequence may include parameters for generating RF pulses (e.g., parameters identifying transmit duration, waveform, amplitude, phase, etc.), parameters for generating gradient fields (e.g., parameters identifying transmit duration, waveform, amplitude, phase, etc.), timing parameters governing when RF or gradient pulses are generated or when the receive coil(s) are configured to detect MR signals generated by the subject, among other functionality. In some embodiments, a pulse sequence may include parameters specifying one or more navigator RF pulses, as described herein.

Examples of pulse sequences include zero echo time (ZTE) pulse sequences, balance steady-state free precession (bSSFP) pulse sequences, gradient echo pulse sequences, inversion recovery pulse sequences, DWI pulse sequences, spin echo pulse sequences including conventional spin echo (CSE) pulse sequences, fast spin echo (FSE) pulse sequences, turbo spin echo (TSE) pulse sequences or any multi-spin echo pulse sequences such a diffusion weighted spin echo pulse sequences, inversion recovery spin echo pulse sequences, arterial spin labeling pulse sequences, and Overhauser imaging pulse sequences, among others.

As illustrated in FIG. 1, the controller 106 may communicate with the computing device 104, which may be programmed to process received MR data. In a non-limiting example, the computing device 104 may process received MR data to generate one or more MR images using any suitable image reconstruction processes, including the execution of techniques involving the estimation of navigator maps for DWI data, as described herein. Additionally or alternatively, the controller 106 may process received MR data to perform DWI reconstruction based on the techniques described herein. The controller 106 may provide information about one or more pulse sequences to computing device 104 for the processing of data by the computing device. In a non-limiting example, the controller 106 may provide information about one or more pulse sequences to the computing device 104 and the computing device 104 may estimate navigator maps and perform DWI reconstruction based, at least in part, on the provided information.

The computing device 104 may be any electronic device configured to process acquired MR data and generate one or more images of a subject being imaged. The computing device 104 may include at least one processor and a memory (e.g., a processing circuit). The memory may store processor-executable instructions that, when executed by a processor, cause the processor to perform one or more of the operations described herein. The processor may include a microprocessor, an application-specific integrated circuit (ASIC), a field-programmable gate array (FPGA), a graphics processing unit (GPU), a tensor processing unity (TPU), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor may read instructions. The instructions may include code generated from any suitable computer programming language. The computing device 104 may include any or all of the components and perform any or all of the functions of the computer system 1000 described in connection with FIG. 10. In some implementations, the computing device 104 may be located in a same room as the MRI system 100 or coupled to the MRI system 100 via wired or wireless connection.

In some implementations, computing device 104 may be a fixed electronic device such as a desktop computer, a server, a rack-mounted computer, or any other suitable fixed electronic device that may be configured to process MR data and generate one or more images from DWI signals. Alternatively, computing device 104 may be a portable device such as a smart phone, a personal digital assistant, a laptop computer, a tablet computer, or any other portable device that may be configured to process DWI data according to the techniques described herein. In some implementations, computing device 104 may comprise multiple computing devices of any suitable type, as aspects of the disclosure provided herein are not limited in this respect. In some implementations, operations that are described as being performed by the computing device 104 may instead be performed by the controller 106, or vice-versa. In some implementations, certain operations may be performed by both the controller 106 and the computing device 104 via communications between said devices.

The MRI system 100 may include one or more external sensors 178. The one or more external sensors may assist in detecting one or more error sources (e.g., motion, noise) which degrade image quality. The controller 106 may be configured to receive information from the one or more external sensors 178. In some embodiments, the controller 106 of the MRI system 100 may be configured to control operations of the one or more external sensors 178, as well as collect information from the one or more external sensors 178. The data collected from the one or more external sensors 178 may be stored in a suitable computer memory and may be utilized to assist with various processing operations of the MRI system 100.

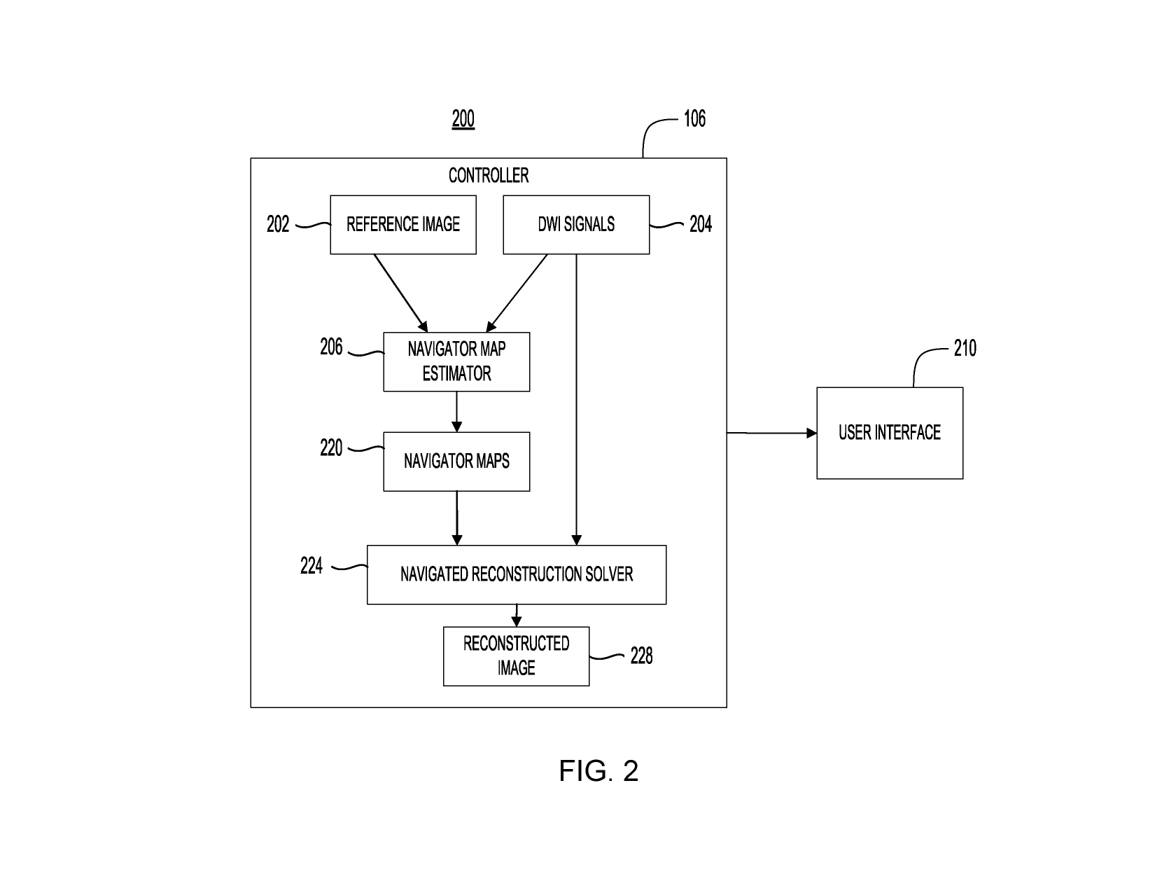

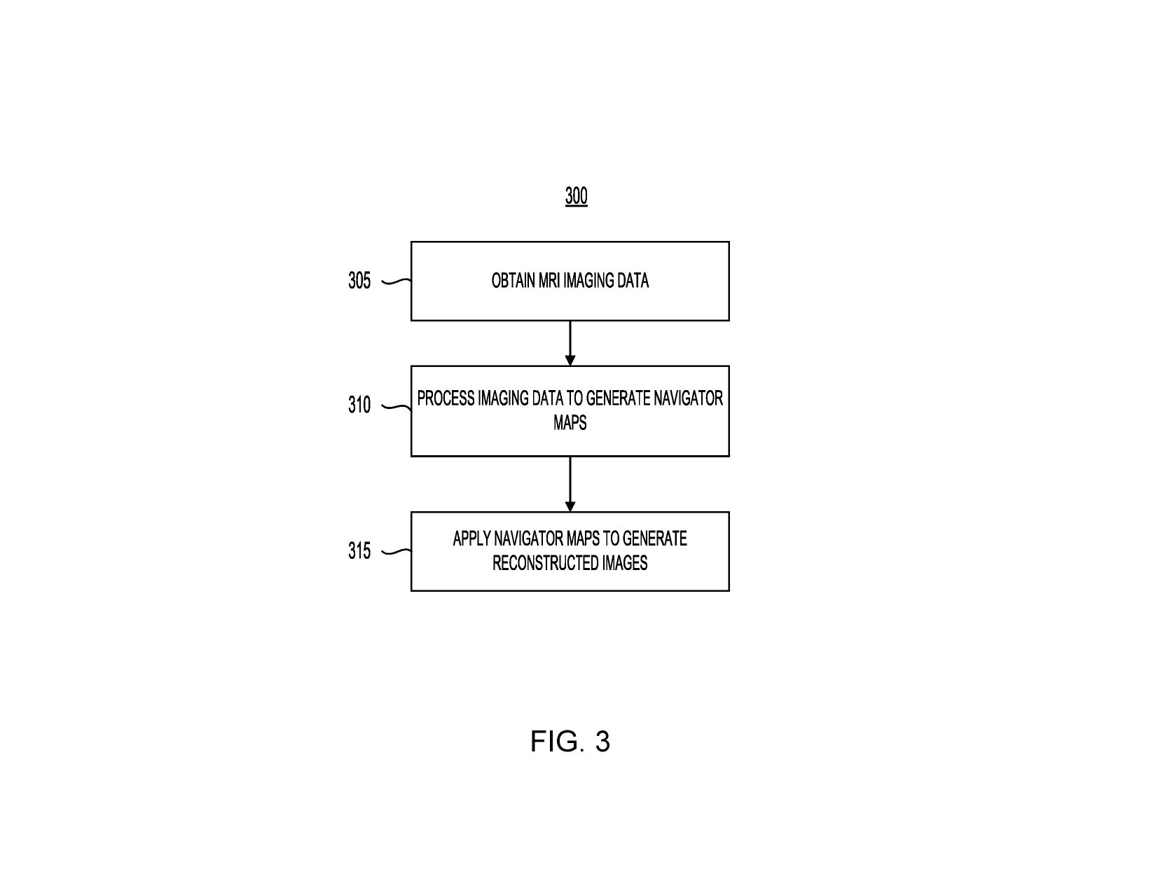

FIG. 2 illustrates an example system 200 for estimating navigator maps for DWI image reconstruction, in accordance with one or more implementations. In a non-limiting example, the system 200 may be used to perform all or part of the example method 300 described in connection with FIG. 3, as well as any other operations described herein. In some implementations, the system 200 forms a portion of an MRI system, such as MRI system 100 described in connection with FIG. 1. In some implementations, the system 200 is external to an MRI system but communicates with the MRI system (or components thereof) to perform the example method 300 of FIG. 3 as described herein.

As shown in FIG. 2, an embodiment of the system 200 may include the controller 106 and a user interface 210. In some implementations, the functionality of the controller 106 as described in connection with FIG. 2 may be implemented on a computing device in communication with the controller 106, such as the computing device 104 described in connection with FIG. 1. The user interface 210 may present or enable inspection of any of the DWI signals, reference images, or reconstructed DWI images described herein. The user interface 210 may provide input relating to the performance of DWI image reconstruction techniques, in a non-limiting example, by receiving input or configuration data relating to the estimation of navigator maps, the performance of MR scans, or the utilization of reference images for a particular subject.

The user interface 210 may allow a user to select a type of imaging to be performed by the MRI system (e.g., diffusion-weighted imaging, etc.), select a sampling density for the MR scan, or to define any other type of parameter relating to MR imaging or model training as described herein. In some implementations, the user interface 210 may display, via a display in communication with the user interface 210, DWI signal data, reference images, or reconstructed DWI data generated using the techniques described herein. The user interface 210 may allow a user to initiate imaging by the MRI system, or to execute or coordinate any of the estimation techniques described herein.

The controller 106 may control aspects of the example system 200, in a non-limiting example to perform at least a portion of the example method 300 described in connection with FIG. 3, as well as any other operations described herein. In some implementations, the controller 106 may control one or more operations of the MRI system, such as the MRI system 100 described in connection with FIG. 1. Additionally or alternatively, the computing device 104 of FIG. 1 may perform some or all of the functionality of the controller 106. In such implementations, the computing device 104 may be in communication with the controller 106 to exchange information as necessary to achieve desired results.

The controller 106 may be implemented using software, hardware, or a combination thereof. The controller 106 may include at least one processor and a memory (e.g., a processing circuit). The memory may store processor-executable instructions that, when executed by a processor, cause the processor to perform one or more of the operations described herein. The processor may include a microprocessor, an ASIC, an FPGA, a GPU, a TPU, etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, ROM, RAM, EEPROM, EPROM, flash memory, optical media, or any other suitable memory from which the processor may read instructions. The instructions may include code generated from any suitable computer programming language. The controller 106 may include any or all of the components and perform any or all of the functions of the computer system 1000 described in connection with FIG. 10.

The controller 106 may be configured to perform one or more functions described herein. The controller 106 may store or capture a reference image 202 of a subject. The reference image 202 may be an image of a subject that captures the same portion as a corresponding set of DWI signals 154 (e.g., which make up a multi-shot interleaved DWI of the same subject). The reference image 202 may be a zero b-value DWI scan image, and may be a complex reference image that may be well aligned with the corresponding set of DWI signals 154. In a non-limiting example, at a low image-resolution level, the reference image 202 may fully (or partially beyond a predetermined threshold) overlap with the multi-shot DWI signals 154 that are to-be-reconstructed using the techniques described herein. In some implementations, the reference image may be an image with different contrast (e.g., T1, T2, proton density, fluid-attenuated inversion recovery (FLAIR), etc.), a combination of reference images, or an image scanned for calibration purposes, among others. In some implementations, the reference image 202 can be optimized for a subject contrast. A given base type of sequence can be optimized to produce different types of contrast. In a non-limiting example, contrast can be optimized by modifying timings within the sequence like TE (time echo), TR (repetition time), echo train length, or inversion time, among others. Optimization may also be performed by adding or removing RF pulses, or varying parameters within a sequence. Such techniques may be utilized to create different types of image contrast to using the same basic kind of sequence. Non-limiting examples of sequences include T1-weighted, T2-weighted, FLAIR, water/fat separated, fat suppressed, phase contrast, or blood contrast, among others. Contrast in MRI refers to the relative image intensities of different tissues (e.g., white matter, gray matter). Such intensities are controlled by the image sequence used (e.g., DWI, T1, T2, etc.) to manifest different pathologies to-be diagnosed.

The reference image may be utilized in connection with the techniques described herein to estimate navigator maps for the set of DWI signals 204. The set of DWI signals 204 may include shots of a multi-shot interleaved DWI scan of a subject. The DWI signals 204 may collectively capture a region of a subject that may also be depicted in the corresponding reference image 202. In some implementations, the DWI signals 204 may be acquired using the same coil sensitivity profile as that of the reference image 204. This may apply to virtual coil sensitivity profiles obtained from, coil compression and pre-whitening, in a non-limiting example. Multi-shot imaging refers to splitting the acquisition of MRI data (e.g., the DWI data) into multiple readouts. The DWI signals 204 may include data in the k-space (e.g., frequency-domain), which may be reconstructed using the techniques described herein to obtain a spatial-domain (or “image domain”) image. Multi-shot DWI signals 204 may be utilized to image a subject when a sufficient portion of k-space data cannot be fully covered by a single readout (single-shot).

Multi-shot DWI images divide the image into segments, acquiring one at a time and once per shot. This composition scheme for the DWI signals 204 may be vulnerable to motion. In a non-limiting example, if the subject being imaged at a different state for each shot, the composed "sufficient" portion could be a mixture, and may leave artefacts in the reconstructed image. Typical patient motion may include gross motion of the body part being imaged, physiological motion, or motion of distal parts of the body (reaching into the imaging field of view, in a non-limiting example), among others. Besides motion, other potential causes of phase variation across shots of the DWI signals 204 include changes in the scanner magnetic or RF fields (e.g., eddy currents, hysteresis, movement of metal objects in or near the scanner, heating of electronics or magnetic components, etc.). Although the techniques described herein provide examples where the reconstructed multi-shot MR images are DWI MR images, it should be understood that the techniques described herein are not limited solely to multi-shot DWI images, and indeed may be implemented with any type of multi-shot MR image.

The DWI signals 204 may be captured by any suitable MR imaging technique (e.g., via the MR system 100). In a non-limiting example, the DWI signals 204 may be captured using a low-field MR system. In some implementations, pulse sequences such as diffusion-weighted steady state free precession (DW-SSFP) sequences, which may be specifically designed for use or optimal performance in the low-field context, may be utilized to generate the set of DWI signals 204. The set of DWI signals 204 may also be captured using other types of MR imaging systems. Each DWI signal of the set of DWI signals 204 may correspond to a single shot of multi-shot interleaved DWI. Each shot of the set of DWI signals 204 may correspond to a high b-value image (as a non-limiting example, a b-value that may be greater than the b-value of the reference image 202). Both the set of DWI signals 204 and the corresponding reference image 202 may be provided as input to the navigation map estimator 206, which may generate a respective navigator map for each shot in the set of DWI signals 204. The reference image 202 may have a different phase than the DWI signals 204.

The controller 106 may include a navigator map estimator 206, which may estimate a navigator map for each shot in the set of DWI signals 204 using the reference image 202. The navigator map estimator 206 may be implemented in hardware, software, or a combination of hardware and software. The navigator map estimator 206 may generate the navigator maps 220 using the techniques described herein, which may include a phase component and a magnitude component. The navigator maps 220 and the DWI signals 204 may be provided as input to the navigated reconstruction solver 224, which may apply the estimated navigator maps 220 to the DWI signals 204 to generate the reconstructed image 228. The navigator maps 220 may be similar to the estimated navigator maps 220 or the low-resolution maps of estimated motion phase and magnitude 320 described in connection with FIGS. 2 and 3, respectively. The navigator maps 220 may be generated using the techniques described herein.

To estimate the navigator maps 220, the navigator map estimator 206 may perform a regularized optimization to estimate the ratio image of the reference image and a respective shot of the DWI signals 204 (e.g., the voxel-wise division of both images). This image domain voxel-wise relation may also be compactly captured in the k-space as a convolution. The estimation of the navigator maps 220 is performed as a regularized minimization problem. The magnitude map of the estimated navigator maps may be utilized for detecting shots with excessive motion that should be rejected and re-acquired. The estimation process for the navigator maps 220 is further described herein. In some implementations, the DWI signals 204 may themselves indicate excessive motion for shots to be rejected. In some implementations, data from the external sensors 178 may indicate excessive motion (e.g., vibration sensors, accelerometers, gyroscopes, etc.). Excessive motion may be any amount of motion or acceleration that exceeds a predetermined or configured threshold.

Respective navigator maps 220 may be estimated for each shot in the DWI signals 204. The phase component of the navigator maps 220 may be applied to the DWI signals 204 by the navigated reconstruction solver 224 to generate the reconstructed image 228. The navigated reconstruction solver 224 may include hardware, software, or combinations of hardware and software, as described in detail herein, to generate the reconstructed image 228. Motion-phase may be primarily a concept of DWI. In the context of DWI, depending on the extent of imaging subject movements, motion may be categorized as either bulk motion or small motion. Small motion refers to movements that are sub-pixel or sub-voxel (e.g., movement that may be not great enough to shift into another pixel). As such, for small motion, the pixel value magnitude does not change. The pixel value phase does change due to the physics of DWI. These phase changes cause data inconsistencies for multi-shot DWI, which may be corrected using the navigator maps that are estimated by the navigator map estimator 206 using the techniques described herein.

Once the navigator maps for the DWI signals 204 have been estimated, the navigator map estimator 206 may perform navigated reconstruction to reconstruct images from the DWI signals 204. In the context of DWI, navigated reconstruction refers to reconstructing a DWI image in a way that the motion-phase data inconsistencies are removed or mitigated, such that inconsistencies are less-pronounced in the reconstructed image. The reconstruction techniques performed by the navigated reconstruction solver 224 may be referred as “self-navigated,” because the techniques described herein need not rely on external data (e.g., other instruments to measure the motion-phase map).

Once the navigator maps 220 have been utilized to generate the reconstructed image 228, may the reconstructed image 228 may be stored in memory of the controller 106 or another computing device (e.g., the computing device 104). The reconstructed image 228 may then be presented on the user interface 210 or transmitted to another computing device, in a non-limiting example. The image reconstruction techniques described herein may be self-navigated techniques, because external data (e.g., from external instruments that somehow measure the motion-phase map) may not be required to perform image reconstruction.

. Images from different MRI sequences may intrinsically have different phases. As a consequence, the reference image 202, which may be obtained from a different sequence than the DWI signals 204, may have a different phase from that of the DWI signals 204. This phase difference may be a static phase difference (sometimes referred to as a shot-invariant phase difference). In some implementations, a static phase or magnitude map may be applied to the reference image 202 (e.g., via element-wise multiplication) to account for this shot-invariant phase difference. The navigator maps 220 generated by the navigator map reconstruction solver 224 may be utilized to correct for shot-variant phase differences between the reference image 202 and the DWI signals 204.

FIG. 3 depicts an example process 300 for imaging a subject using an MRI system as disclosed herein. Once a subject is positioned in an imaging region of the MRI system, the MRI system can be used to acquire imaging data (305). The imaging data may be or may include DWI encoded signals. The imaging data can be processed to generate navigator maps (310). The navigator maps can be applied in the reconstruction of the input imaging data to produce reconstructed images (315).

The method 300 may be executed using any suitable computing system (e.g., the controller 106, the computing device 104 of FIG. 1, the computing system 1000 of FIG. 10, etc.). It may be appreciated that certain steps of the method 300 may be executed in parallel (e.g., concurrently) or sequentially, while still achieving useful results. The method 300 may be executed iteratively to generate navigator maps for each shot of a multi-shot interleaved diffusion-weighted image, as described herein.

The method 300 may include act 305, in which MRI imaging data of a subject may be obtained. The imaging data may comprise a reference image (e.g., the reference image 202, the well-aligned structural image 402, etc.) The reference image may be obtained, in a non-limiting example, by capturing a structural image of a region of the subject on which a DWI scan will be performed. In a non-limiting example, the reference image may be an image of a subject that captures the same portion as a corresponding set of DWI signals (e.g., which make up a multi-shot interleaved DWI of the same subject). In some implementations, the reference image may be a zero b-value DWI scan image, and may be a complex reference image that may be well aligned with the corresponding set of DWI signals. In a non-limiting example, DWI scans in practice may include protocols with a structural imaging sequence, such as a zero b-value DWI scan. The zero b-value DWI scan provide a zero b-value image, which may be utilized as the reference image. The reference image may also be an image of the subject with different contrast (e.g., T1, T2, proton density, FLAIR), a combination of reference images, or may be an image scanned for calibration purposes. In some implementations, the reference image may be obtained (e.g., received, retrieved) from an external computing system or database.

Act 305 may also include capturing a multi-shot DWI image of the subject. Multi-shot images divide the DWI image portion into segments, acquiring one at a time and once per shot. Such multi-shot DWI images are vulnerable to motion. In a non-limiting example, if the subject being imaged is at a different state for each shot, the composed "sufficient" portion is a mixture, and may leave artefacts in the reconstructed image. Typical patient motion may include gross motion of the body part being imaged, physiological motion, or motion of distal parts of the body (reaching into the imaging field of view, in a non-limiting example), among others. Besides motion, other potential causes of phase variation across shots of the DWI image include changes in the scanner magnetic or RF fields (e.g., eddy currents, hysteresis, movement of metal objects in or near the scanner, heating of electronics or magnetic components, etc.). The techniques described herein may be utilized to correct the phase variation across shots.

The multi-shot DWI image may be captured by any suitable MR imaging technique (e.g., via the MR system 100). In a non-limiting example, the DWI image may be captured using a low-field MR system. In some implementations, pulse sequences such as diffusion-weighted steady state free precession (DW-SSFP) sequences, which may be specifically designed for use or optimal performance in the low-field context, may be utilized to capture or acquire the DWI image. The multi-shot DWI image may also be captured using other types of MR imaging systems. Each shot of the multi-shot DWI image may include a high b-value image (e.g., a b-value that may be greater than the b-value of the reference image). A high b-value may be a range of b-values from 100 to 2000, or any b-value that may be greater than zero, in a non-limiting example. The DWI image may be acquired separately from the reference image, or during the same procedure in which the reference image is captured. In some implementations, the multi-shot DWI image may be obtained (e.g., received, retrieved) from an external computing system or database. In some implementations, the acquisition of the DWI image may utilize the same coil sensitivity profile as that used to acquire the reference image.

The method 300 may include act 315, in which navigator maps for each shot of the DWI image are generated. A respective navigator map may be generated for each shot of the multi-shot DWI image acquired in act 310. The navigator maps may be utilized in phase-navigated correction of the DWI image, to correct for shot-variant motion phase in each shot of the DWI image. In some embodiments, prior to estimating the navigator maps for each shot, a shot-invariant phase difference map is estimated between the reference and the multi-shot interleaved DWI image, which may be subsequently applied to either the reference image or the multi-shot DWI such that both data have the same shot-invariant phase.

In addition to the shot-varying motion-induced phase differences, the DWI image to be corrected may contain additional phase that may be static across shots. In a non-limiting example, uncompensated DWI encoding gradient moments or eddy-current fields may generate a (spatially-varying) static phase difference with respect to the structural reference image used for estimation. The static phase map may be obtained with various different approaches (e.g., non-navigated reconstruction, gridding, machine-learning models, etc.). The static phase difference map may then be used prior to navigator map estimation to modulate the structural reference image, allowing the proposed method to focus on estimating the phase differences specifically due to temporally varying motion induced phase corruption. Similar to including a shot-invariant static phase map for improving the robustness, instead of using structural images, in some implementations the estimation may be initiated from a synthesized high b-value image obtained from, in a non-limiting example, a deep learning reconstruction method. The reference image may also be synthesized from a combination of other previously acquired images.

The shot-invariant phase difference map (sometimes referred to herein as the static phase and magnitude map) is estimated via non-navigated reconstruction of k-space data from partial or all shots of the multi-shot DWI image. Non-navigated reconstruction may be any reconstruction technique that does not involve navigated reconstruction, such as gridding or machine-learning techniques (e.g., trained convolutional neural network models, etc.), among others. Once estimated, the shot-invariant phase difference map may be applied (e.g., by element-wise multiplication or convolution, etc.) to either the reference image or the multi-shot DWI such that both data have the same shot-invariant phase. The DWI image and reference image, once having the same shot-invariant phase, may be utilized to estimate the navigator maps.

As described herein, the navigator maps may be estimated by minimizing a difference between data of the reference image convolved with the navigator map and a respective shot of the plurality of shots. This approach is described herein in connection with Formulations 2 and 3, in which regularizers and regularization terms are utilized to the phase variations between the images, which allows for calculation of navigator maps from undersampled data (e.g., captured from a low-field MR system, etc.). Any suitable regularizer may be utilized in performing the regularization process. The regularizer may be executed according to a set of regularization terms, as described herein, to estimate the navigator maps. Formulation 2, as described herein, may be utilized to implement an image-domain regularized minimization technique that generates the navigator maps, and Formulation 3 may be utilized to implement a frequency-domain regularized minimization technique that generates the navigator maps. In some implementations, if the reference image might be in the image domain, the reference image may be transformed into the frequency domain (e.g., utilizing a Fourier transform) prior to executing the regularized minimization techniques described herein.

In some implementations, the output of the regularized minimization process includes generation of an estimated convolutional kernel, which represents the estimated navigator map for a particular shot in the frequency domain. To generate the navigator map, the estimated convolutional kernel may be transformed into the image domain (e.g., by performing an inverse-Fourier transform. A motion phase map may be extracted as the phase component from the navigator map, and utilized to correct shot-variant phase motion in the multi-shot interleaved DWI image. A corresponding to the magnitude map may be extracted as the magnitude component from the navigator map. The magnitude component may be utilized to reject shots that include excessive motion, and the motion phase map may be utilized to correct shot-variant phase motion in each shot of the acquired DWI image.

In some implementations, the navigator maps may be estimated in a hybrid image-frequency domain. The hybrid image-frequency domain may include any intermittent domain after incomplete multi-dimensional Fourier transform. The images described herein may be multi-dimensional (e.g., 2D, 3D images). Multi-dimensional Fourier transform processes (such as those described herein) are separable processes. In a non-limiting example, for a 3D spatial domain (with Cartesian directions x, y, and z), the multi-dimensional Fourier transform may be performed by doing single-dimensional Fourier transforms along each direction sequentially (or in parallel). An incomplete multi-dimensional Fourier transform involves not completing FT along all directions (e.g., leaving at least one dimension un-transformed). To achieve a hybrid approach, the estimation process described in connection with Formulation 3 may be utilized, but utilizing an incomplete Fourier transform F as part of the Fourier transform identity.

The method 300 may include act 315, in which it may be determined whether any shots should be rejected based on the navigator maps. As described herein, the respective magnitude portion of the navigator maps estimated for each shot may be utilized to reject certain shots of the DWI image that have excessive motion. In a non-limiting example, if a magnitude map indicates a magnitude value or group of magnitude values that exceed a predetermined threshold (e.g., defined via a configuration file or user input, etc.), the respective shot may be marked as rejected. In act 315, in which image reconstruction may be performed using the navigator maps. To do so, the navigator maps may be applied to each respective shot of the multi-shot DWI image to correct motion or system induced image phase. Applying the navigator maps may be performed by elementwise multiplication or by convolution. The navigator maps may be utilized to perform navigated image reconstruction of k-space data from all shots of the multi-shot interleaved DWI using the estimated navigator maps. In some implementations, the motion phase map of the navigator map may be utilized to perform the reconstruction, rather than the entirety of the navigator map. The reconstructed DWI image may then be stored, provided for display, provided to another computing system, or subjected to further processing operations.

The techniques described herein may be utilized for any type of image encoding, and therefore may be utilized with any type of 2D, 3D, or other type of multidimensional imaging process. As described herein, a hybrid data layout may be utilized, for instance, in 3D acquisitions that are regularly sampled along the readout dimension, an inverse Fourier transform may be applied along the readout dimension. The estimation of the navigator maps, as described herein, may then be conducted on the resulting 2D k-space data. These approaches are therefore independent of both the navigator acquisition strategy and image encoding due to the use of a separate reference image. Additionally, the techniques described herein may inherently handle contrast differences between the reference image and the DWI image that may be to be corrected. The source of the structural reference image may image may be imaging data where motion sensitivity may be minimized.

These approaches described herein may further be utilized with aggressively undersampled data (e.g., a single shot out of ~500) to estimate a navigator map. The use of a separate reference image may also be utilized for bulk motion correction. Processing efficiency may be improved by generating the navigator maps in parallel with the multi-shot DWI image acquisition process, because the estimation process does not require knowledge of the full high b-value k-space data of the full DWI image. Performing the estimation process in parallel may shorten the waiting time between the end of the data acquisition and the start of the model based high b-value image reconstruction using the motion-phase maps. Further, corrections to the image acquisition may be calculated and applied during the acquisition, including updating the image acquisition field of view or orientation to match bulk motion of the subject or determining whether to reject or reacquire DWI image shots (e.g., segments).

The estimation of the motion phase map per segment of DWI data may be improved by utilizing an image consistency metric. This metric may be a combination of consistency between segments of correction, and between segments and reference image (all of which are imaging the same portion of the subject). Note that while the various examples described herein provide techniques for a multi-shot 3D spin echo DWI sequence, the same techniques may be applied to correct any multi-shot 2D or 3D imaging sequence with a reference image.

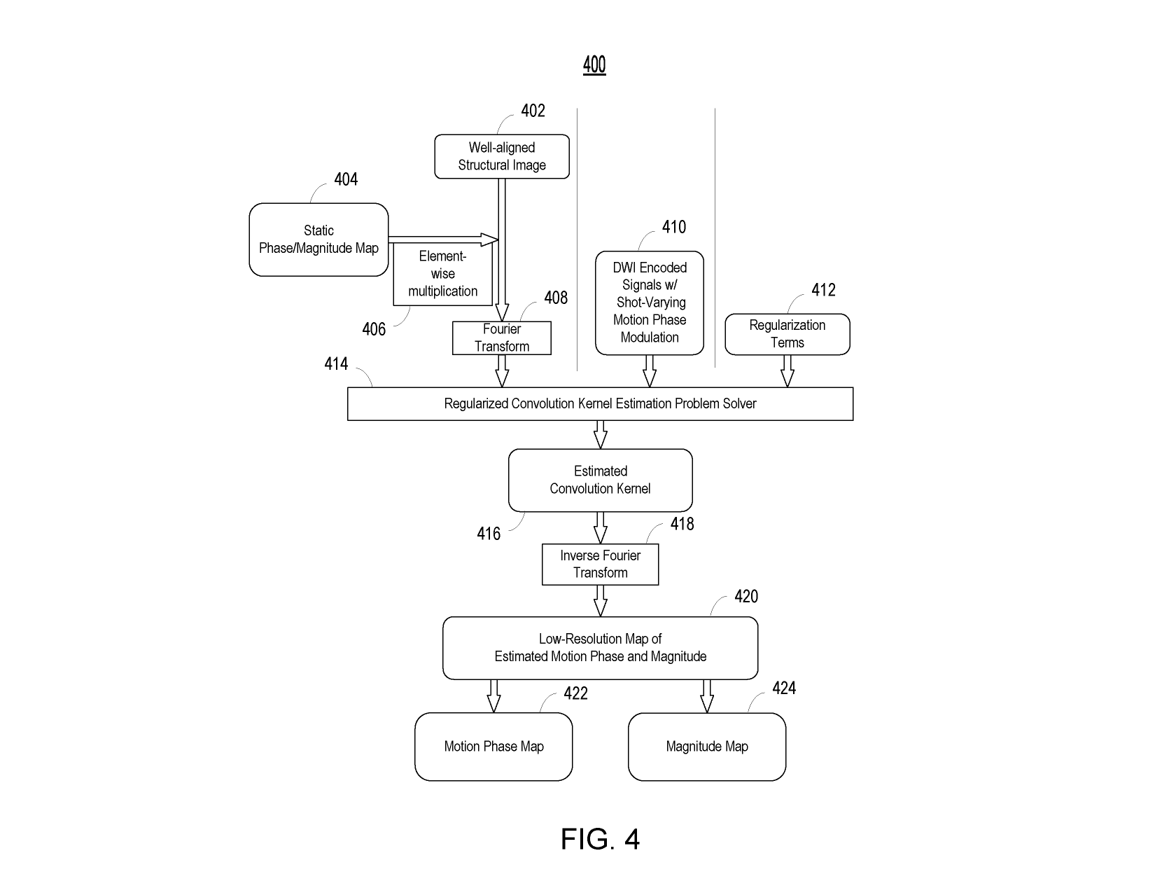

FIG. 4 depicts an example dataflow diagram 400 of a process for estimating navigator maps for DWI image reconstruction based on k-space data, in accordance with one or more implementations. The estimation process shown in the diagram 400 may be performed, in a non-limiting example, by any computing device described herein, such as the controller 106, computing device 104, or the computer system 1000, among others. The estimation process shown in the diagram 400 may be utilized to estimate navigator maps 420, which may be used to reconstruct diffusion-weighted images or to determine whether certain shots of a multi-shot interleaved diffusion-weighted image must be reacquired for proper reconstruction, as described herein.

The well-aligned structural image 402, along with the DWI encoded signals 410 (e.g., which include shot-varying motion-phase modulation that may be to be corrected) may be utilized in connection with regularization terms 412 (e.g., the terms described in connection with Formulation 4) to estimate a convolutional kernel 416 in the k-space. The convolutional kernel 416 may then applied to an inverse-Fourier transform 418 to generate the low-resolution map of estimated motion-phase and magnitude 420 (e.g., the estimated navigator map 220).

The well-aligned structural image 402 may be any image-domain reference image of a subject (e.g., the reference image 202, the reference image 152) that fully (or partially beyond a predetermined threshold) overlaps with the DWI encoded signals 410, as described herein. An optional static phase/magnitude map 404 may be applied to the well-aligned structural image 402 using an element-wise multiplication process 406. The well-aligned structural image 402, which includes data in the image domain, may be subjected to a Fourier transform 408 (e.g., a fast Fourier transform (FFT), other Fourier transform processes, etc.) to generate k-space data of the well-aligned structural image 402 in the frequency domain. The DWI encoded signals 410 may also include k-space data for multiple shots of a DWI image that may be to be corrected. The DWI encoded signals 410 may be suitable DWI data, such as high b-value image data in the frequency domain (e.g., k-space data). The DWI encoded signals 410 include shot-varying motion-phase modulation, as described herein. The DWI encoded signals 410 may correspond to a DWI image of the same subject as the well-aligned structural image 402.

The regularized convolution kernel estimation problem solver 414 may be executed by a suitable computer system (e.g., the controller 106, the computing device 104, the computer system 1000, etc.) to estimate the convolutional kernel 416. The convolutional kernel 416 may a corresponding low-resolution map of the estimated motion phase and magnitude 420 represented in the frequency domain (e.g., k-space). The regularized convolution kernel estimation problem solver 414 may estimate the convolutional kernel 416 using the frequency-domain version of the well-aligned structural image 402 and the DWI encoded signals 410, as described herein. In a non-limiting example, the regularized convolution kernel estimation problem solver 414 may estimate the convolution kernel 416 using Formulation 4, as described herein. The regularization terms 412 may be any of the regularization terms described herein, and may be utilized in connection with the regularizer chosen to optimize the convolution kernel 416.

Once the convolution kernel 416 has been estimated using the techniques described herein, an inverse-Fourier transform 418 (e.g., with zero-padding, as needed) may be applied to the convolution kernel 416 to generate the estimated motion phase and magnitude map 420 (e.g., the navigator map 220, etc.). The motion-phase portion of the estimated motion phase and magnitude map 420 (the motion phase map 422) may be utilized in Formulation 5 to perform reconstruction of the DWI encoded signals 410 to generate a corrected DWI image. The magnitude portion of the estimated motion phase and magnitude map 420 (the magnitude map 424) may be utilized to reject certain shots of the DWI signals 410 that have excessive motion (e.g., those that indicate motion above a predetermined threshold).

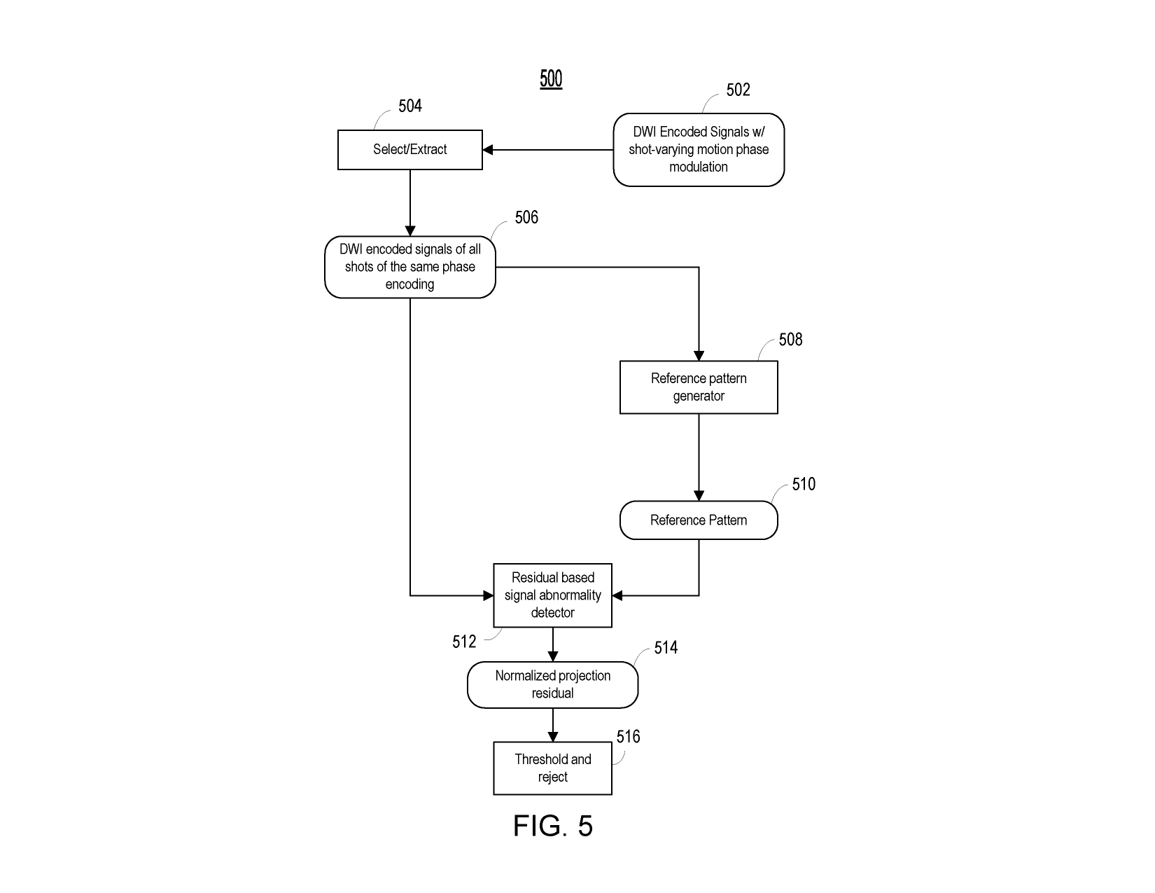

FIG. 5 depicts a flowchart of an example method 500 of rejecting imaging data. Raw DWI encoded signals 502 may be obtained from MRI imaging of a subject. DWI encoded signals of the same phase encoding 506 may be selected and extracted (504). For example, DWI encoded signals with zero-phase encoding (k-space center) may be selected then extracted from a set of DWI encoded signals. DWI encoded signals of the same phase encoding 506 may be used as input to a reference pattern generator 508 which may output a reference pattern 510 for motion level quantification. Reference pattern 510 and DWI encoded signals 506 may be used as input to a residual based signal abnormality detector 512, which may generate a residual. The residual may be projected to a sub-space formed from the reference pattern 510 and normalized to obtain normalized projection residual 514. If the projection residual 514 has a normalized residual energy level above a chosen threshold (e.g., a threshold of 0.1), the data from that shot/segment of the image reconstruction may be rejected (516).

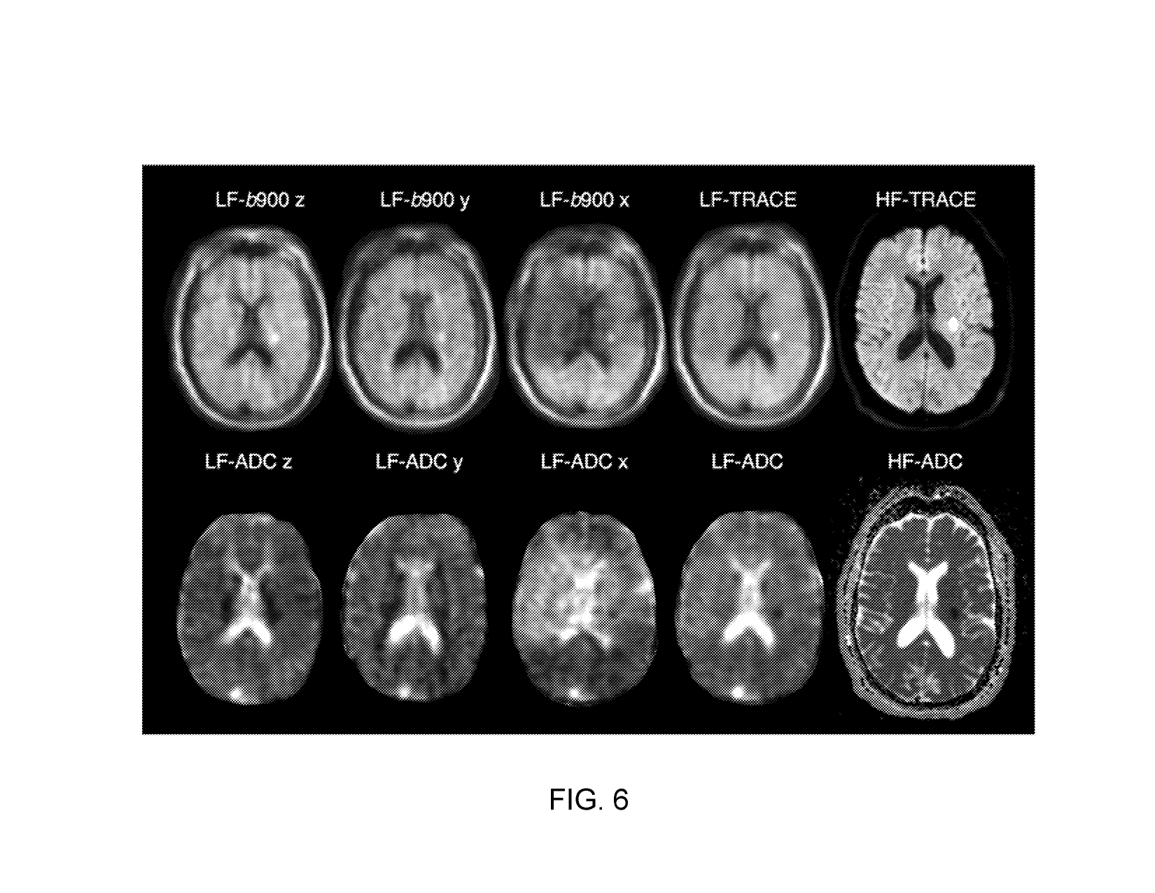

FIG. 6 depicts MRI imaging data of an example of each diffusion direction individually, the combined TRACE weight, and ADC maps compared with conventional high-field (HF) MRI acquired within 30 minutes of the low-field (LF) acquisition.

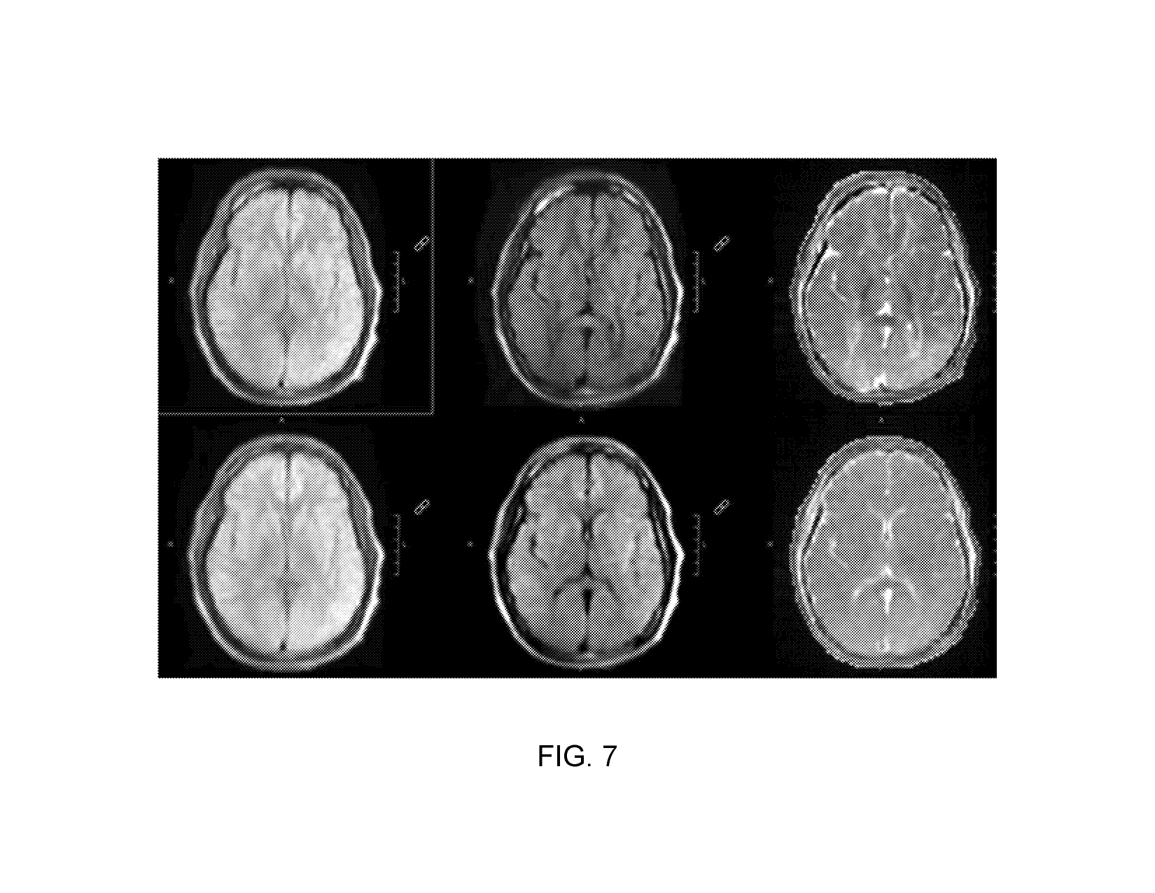

FIG. 7 depicts MRI imaging data of an example of single direction diffusion (top row) compared to multi-direction diffusion (bottom row). Diffusion weighted b=900 images from the single-direction diffusion (center, top) show white matter anisotropy contrast that is reduced in the multi-direction b=900 image (center, bottom). Similarly the ADC images (right column) show this effect.

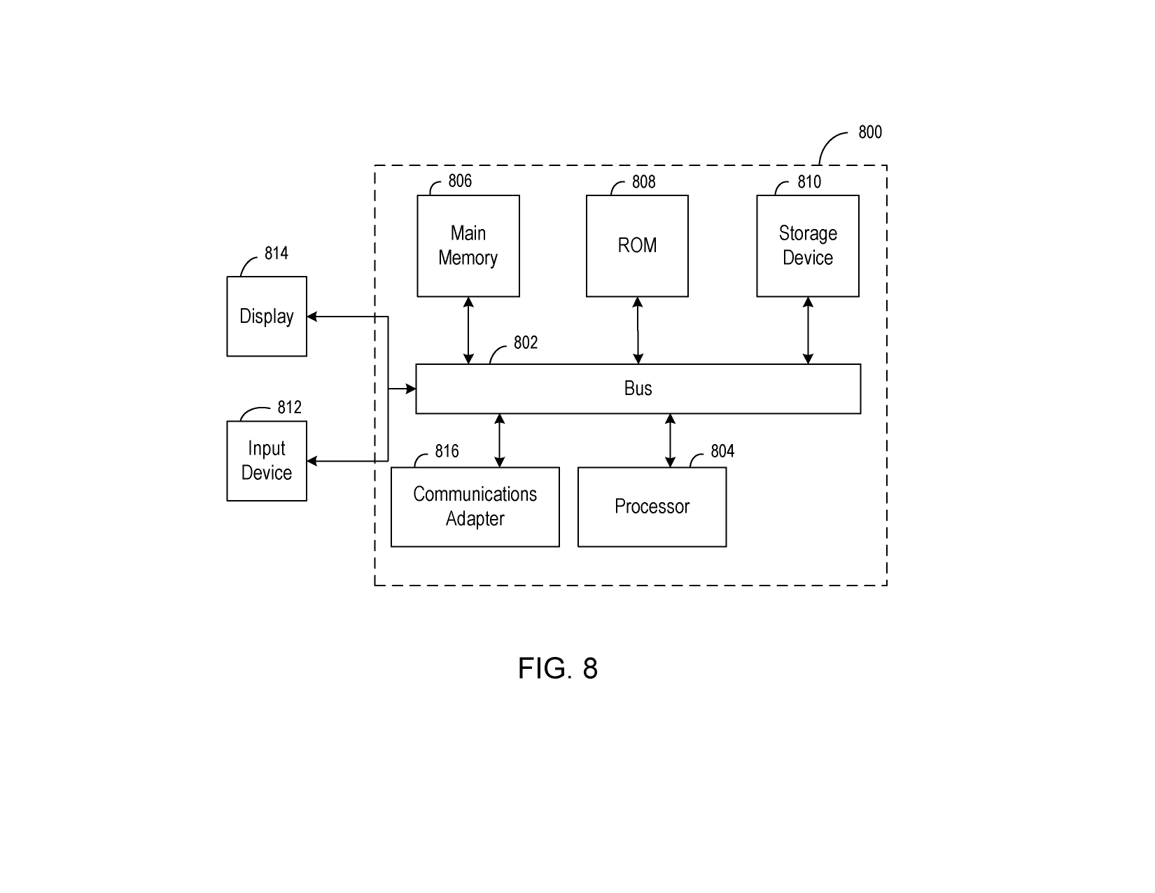

FIG. 8 illustrates a component diagram of an example computing system suitable for use in the various implementations described herein, according to an example implementation. In a non-limiting example, the computing system 800 may implement a computing device 104 or controller 106 of FIG. 1, or various other example systems and devices described in the present disclosure.

The computing system 800 includes a bus 802 or other communication component for communicating information and a processor 804 coupled to the bus 802 for processing information. The computing system 800 also includes main memory 806, such as a RAM or other dynamic storage device, coupled to the bus 802 for storing information, and instructions to be executed by the processor 804. Main memory 806 may also be used for storing position information, temporary variables, or other intermediate information during execution of instructions by the processor 804. The computing system 800 may further include a ROM 808 or other static storage device coupled to the bus 802 for storing static information and instructions for the processor 804. A storage device 810, such as a solid-state device, magnetic disk, or optical disk, is coupled to the bus 802 for persistently storing information and instructions.

The computing system 800 may be coupled via the bus 802 to a display 814, such as a liquid crystal display, or active matrix display, for displaying information to a user. An input device 812, such as a keyboard including alphanumeric and other keys, may be coupled to the bus 802 for communicating information, and command selections to the processor 804. In another implementation, the input device 812 has a touch screen display. The input device 812 may include any type of biometric sensor, or a cursor control, such as a mouse, a trackball, or cursor direction keys, for communicating direction information and command selections to the processor 804 and for controlling cursor movement on the display 814.

In some implementations, the computing system 800 may include a communications adapter 816, such as a networking adapter. Communications adapter 816 may be coupled to bus 802 and may be configured to enable communications with a computing or communications network or other computing systems. In various illustrative implementations, any type of networking configuration may be achieved using communications adapter 816, such as wired (e.g., via Ethernet), wireless (e.g., via Wi-Fi, Bluetooth), satellite (e.g., via GPS) pre-configured, ad-hoc, LAN, WAN, and the like.

According to various implementations, the processes of the illustrative implementations that are described herein may be achieved by the computing system 800 in response to the processor 804 executing an implementation of instructions contained in main memory 806. Such instructions may be read into main memory 806 from another computer-readable medium, such as the storage device 810. Execution of the implementation of instructions contained in main memory 806 causes the computing system 800 to perform the illustrative processes described herein. One or more processors in a multi-processing implementation may also be employed to execute the instructions contained in main memory 806. In alternative implementations, hard-wired circuitry may be used in place of or in combination with software instructions to implement illustrative implementations. Thus, implementations are not limited to any specific combination of hardware circuitry and software.

Various potential non-limiting embodiments and aspects of the disclosure include the following:

Embodiment A1: A low-field multi-directional diffusion-weighted magnetic resonance imaging process, the method comprising: quantifying motion based on signal variation; and generating one or more output images based on multi-directional image reconstructions that exclude data based on the quantified motion.

Embodiment A2: The method of Embodiment A1, comprising acquiring imaging data.

Embodiment A3: The method of either Embodiment A1 or A2, wherein imaging data is acquired using a low-field MRI system.

Embodiment A4: The method of any of Embodiments A1 – A3, wherein imaging data is acquired using a low-field MRI system with a field strength no greater than 0.2 T.

Embodiment A5: The method of any of Embodiments A1 – A4, wherein imaging data is acquired using a portable MRI system.

Embodiment A6: The method of any of Embodiments A1 – A5, wherein imaging data is acquired using a portable MRI system that is cartable between locations.

Embodiment A7: The method of any of Embodiments A1 – A6, wherein imaging data is acquired using a portable MRI system that is cartable between locations using one or more battery-powered motors coupled to a set of wheels.

Embodiment A8: The method of any of Embodiments A1 – A7, wherein acquiring imaging data comprises performing a pseudo-trace acquisition encoded along a plurality of axes.

Embodiment A9: The method of any of Embodiments A1 – A8, wherein the multi-directional image reconstructions correspond to a plurality of directions.

Embodiment A10: The method of any of Embodiments A1 – A9, wherein the multi-directional image reconstructions correspond to a first direction and a second direction that is different from the first direction.

Embodiment A11: The method of any of Embodiments A1 – A10, wherein the multi-directional image reconstructions correspond to a first direction that is orthogonal to a second direction.

Embodiment A12: The method of any of Embodiments A1 – A11, wherein the multi-directional image reconstructions correspond to a plurality of directions that are orthogonal to each other.

Embodiment A13: The method of any of Embodiments A1 – A12, wherein the multi-directional image reconstructions correspond to three directions or at least three directions or more than three directions.

Embodiment A14: The method of any of Embodiments A1 – A13, comprising aligning navigator signal across all segments.

Embodiment A15: The method of any of Embodiments A1 – A14, comprising generating a reference pattern.

Embodiment A16: The method of any of Embodiments A1 – A15, comprising generating a reference pattern from an aligned navigator signal for motion level quantification.

Embodiment A17: The method of any of Embodiments A1 – A16, comprising computing normalized projection residual against a reference pattern.

Embodiment A18: The method of any of Embodiments A1 – A17, comprising determining, for each segment, to reject data from the segment.

Embodiment A19: The method of any of Embodiments A1 – A18, comprising determining for each segment, based on a projection having a normalized residual energy level above a chosen threshold, to reject data from the segment.

Embodiment B1: A method of magnetic resonance (MR) imaging using a low-field MR imaging (MRI) system, the method comprising: performing, using an MRI system, a multi-shot, diffusion weighted acquisition with diffusion-encoding along a plurality of physical axes to obtain imaging data of a subject in an imaging region of the MRI system, the imaging data comprising a plurality of shot segments.

Embodiment B2: The method of Embodiment B1, further comprising: performing navigator-based motion rejection.

Embodiment B3: The method of either Embodiment B1 or B2, wherein performing the navigator-based motion rejection comprises: aligning a navigator signal across the shot segments in the multi-shot acquisition to obtain an aligned navigator signal; quantifying motion in the multi-shot acquisition; and rejecting data from shot segments based at least in part on the quantified motion;

Embodiment B4: The method of any of Embodiments B1 – B3, wherein quantifying motion in the multi-shot acquisition comprises computing a normalized projection residual against a reference pattern.

Embodiment B5: The method of any of Embodiments B1 – B4, further comprising independently reconstructing a diffusion-weighted image corresponding to each of the plurality of axes to obtain a plurality of reconstructed images.

Embodiment B6: The method of any of Embodiments B1 – B5, further comprising providing one or more output images based on the independently-reconstructed diffusion-weighted images.

Embodiment B7: The method of any of Embodiments B1 – B6, wherein the one or more output images is provided to at least one of (i) a display device or a computing device for presentation of the one or more output images, (ii) a non-transitory non-volatile computer-readable storage medium for storage of the one or more output images, and/or (iii) a computing system for subsequent accessibility of the one or more output images via a network.

Embodiment B8: The method of any of Embodiments B1 – B7, wherein the plurality of physical axes comprises any combination of an x-axis, a y-axis, and/or a z-axis.

Embodiment B9: The method of any of Embodiments B1 – B8, wherein the one or more output images are based at least in part on (i) a first image without diffusion weighting and (ii) the independently-reconstructed diffusion-weighted images.

Embodiment B10: The method of Embodiment B9, wherein the first image is a b=0 s²/mm image and the diffusion-weighted images are b=900 s²/mm images.

Embodiment B11: The method of any of Embodiments B1 – B10, wherein generating the one or more output images comprises producing an apparent diffusion coefficient (ADC) map based at least in part on the plurality of reconstructed images.

Embodiment B12: The method of Embodiment B11, wherein the ADC map is based at least in part on a pseudo-trace obtained using the plurality of reconstructed images.

Embodiment B13: The method of Embodiment B12, wherein the pseudo-trace is based at least in part on an average of the plurality of reconstructed images.

Embodiment B14: The method of Embodiment B11, wherein the ADC map is produced using a pseudo-trace and an image without diffusion-weighting.

Embodiment B15: The method of any of Embodiments B1 – B14, wherein the one or more output images do not use rejected data.

Embodiment B16: The method of any of Embodiments B1 – B15, wherein the MRI system is a low-field MRI system.

Embodiment B17: The method of any of Embodiments B1 – B16, wherein the MRI system is a portable MRI system.

Embodiment C1: A low-field multi-directional diffusion-weighted magnetic resonance imaging (MRI) process, the method comprising quantifying motion based on signal variation, rejecting data based on the quantified motion, and generating one or more output images based on multi-directional image reconstructions that exclude rejected data.

Embodiment C2: The method of Embodiment C1, further comprising acquiring imaging data by performing a pseudo-trace acquisition encoded along a plurality of physical axes.

Embodiment C3: The method of either Embodiment C1 or C2, further comprising aligning a phase of a navigator signal across all segments.

Embodiment C4: The method of any of Embodiments C1 – C3, further comprising generating a reference pattern from an aligned navigator signal for motion level quantification.

Embodiment C5: The method of any of Embodiments C1 – C4, further comprising projecting the aligned navigator signals to a sub-space corresponding to a reference pattern.

Embodiment C6: The method of any of Embodiments C1 – C5, further comprising determining for each segment, based on a projection having a normalized residual energy level above a chosen threshold, to reject data from the segment.

Embodiment D1: A magnetic resonance imaging (MRI) system comprising one or more processors and being configured to perform any of the methods of any of Embodiments A1 – C6.

Embodiment D2: The MRI system of D1, wherein the MRI system is a low-field MRI system that has a field strength no greater than 0.2 T.

Embodiment D3: The MRI system of either D1 or D2, wherein the MRI system is a portable MRI system that is transportable between locations.

Embodiment D4: The MRI system of any of Embodiment D1 – D3, wherein the MRI system is cartable between locations using one or more battery-powered motors coupled to a set of wheels.

Embodiment D5: The MRI system of any of Embodiment D1 – D4, wherein the MRI system is communicably coupled with a portable computing device.

Embodiment D6: The MRI system of Embodiment D5, wherein the MRI system is configured to receive commands from the portable computing device for any functions performable by the MRI system, and/or the MRI system is configured to provide data and/or images to the computing device for processing and/or review.

Embodiment E1: A non-transitory computer-readable medium comprising computer-executable instructions which, when executed by one or more processors of an MRI system and/or other computing system, causes the MRI system and/or the computing system to perform any of the methods of any of Embodiments A1 – C6.

Embodiment E2: The non-transitory computer-readable medium of Embodiment E1, wherein the instructions are executable by any of the MRI systems of any of Embodiments D1 – D6.

Embodiment E3: The non-transitory computer-readable medium of either Embodiment E1 or E2, wherein the instructions are executable by both an MRI system and a computing system or computing device of any of Embodiments D1 – D6.

Embodiment F1: A low-field MRI system comprising one or more processors configured to perform any of the methods of Embodiments H1 – H18.

Embodiment F2: The MRI system of Embodiment F1, wherein the MRI system has a field strength no greater than 0.2 T.

Embodiment F3: The MRI system of either Embodiment F1 or F2, wherein the MRI system is portable.

Embodiment F4: The MRI system of any of Embodiments F1 – F3, wherein the MRI system is cartable between locations.

Embodiment F5: The MRI system of any of Embodiments F1 – F4, wherein the MRI system is cartable between locations using one or more battery-powered motors coupled to a set of wheels.

Embodiment F6: The MRI system of any of Embodiment F1 – F5, wherein the MRI system is communicably coupled with a portable computing device.

Embodiment F7: The MRI system of Embodiment F6, wherein the MRI system is configured to receive commands from the portable computing device for any functions performable by the MRI system, and/or the MRI system is configured to provide data and/or images to the computing device for processing and/or review.

Embodiment G1: A non-transitory computer-readable medium comprising computer-executable instructions which, when executed by one or more processors of a low-field MRI system and/or other computing system, causes the MRI system and/or the computing system to perform any of the methods of any of Embodiments A1 – C6.

Embodiment G2: The non-transitory computer-readable medium of Embodiment J1, wherein the instructions are executable by any of the MRI systems of any of Embodiments F1 – F7.

Embodiment G3: The non-transitory computer-readable medium of either Embodiment G1 or G2, wherein the instructions are executable by both an MRI system and a computing system or computing device of any of Embodiments F1 – F7.

The implementations described herein have been described with reference to drawings. The drawings illustrate certain details of specific implementations that implement the systems, methods, and programs described herein. Describing the implementations with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “circuit” may include hardware structured to execute the functions described herein. In some implementations, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some implementations, a circuit may take the form of one or more analog circuits, electronic circuits (e.g., integrated circuits (IC), discrete circuits, system on a chip (SOC) circuits), telecommunication circuits, hybrid circuits, and any other type of “circuit.” In this regard, the “circuit” may include any type of component for accomplishing or facilitating achievement of the operations described herein. In a non-limiting example, a circuit as described herein may include one or more transistors, logic gates (e.g., NAND, AND, NOR, OR, XOR, NOT, XNOR), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some implementations, the one or more processors may be embodied in various ways. The one or more processors may be constructed in a manner sufficient to perform at least the operations described herein. In some implementations, the one or more processors may be shared by multiple circuits (e.g., circuit A and circuit B may comprise or otherwise share the same processor, which, in some example implementations, may execute instructions stored, or otherwise accessed, via different areas of memory). Alternatively or additionally, the one or more processors may be structured to perform or otherwise execute certain operations independent of one or more co-processors.

In other example implementations, two or more processors may be coupled via a bus to enable independent, parallel, pipelined, or multi-threaded instruction execution. Each processor may be implemented as one or more general-purpose processors, ASICs, FPGAs, GPUs, TPUs, digital signal processors (DSPs), or other suitable electronic data processing components structured to execute instructions provided by memory. The one or more processors may take the form of a single core processor, multi-core processor (e.g., a dual core processor, triple core processor, or quad core processor), microprocessor, etc. In some implementations, the one or more processors may be external to the apparatus, in a non-limiting example, the one or more processors may be a remote processor (e.g., a cloud-based processor). Alternatively or additionally, the one or more processors may be internal or local to the apparatus. In this regard, a given circuit or components thereof may be disposed locally (e.g., as part of a local server, a local computing system) or remotely (e.g., as part of a remote server such as a cloud based server). To that end, a “circuit” as described herein may include components that are distributed across one or more locations.

An exemplary system for implementing the overall system or portions of the implementations might include a general purpose computing devices in the form of computers, including a processing unit, a system memory, and a system bus that couples various system components including the system memory to the processing unit. Each memory device may include non-transient volatile storage media, non-volatile storage media, non-transitory storage media (e.g., one or more volatile or non-volatile memories), etc. In some implementations, the non-volatile media may take the form of ROM, flash memory (e.g., flash memory such as NAND, 3D NAND, NOR, 3D NOR), EEPROM, MRAM, magnetic storage, hard discs, optical discs, etc. In other implementations, the volatile storage media may take the form of RAM, TRAM, ZRAM, etc. Combinations of the above are also included within the scope of machine-readable media. In this regard, machine-executable instructions comprise, in a non-limiting example, instructions and data, which cause a general-purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components), in accordance with the example implementations described herein.

It should also be noted that the term “input devices,” as described herein, may include any type of input device including, but not limited to, a keyboard, a keypad, a mouse, joystick, or other input devices performing a similar function. Comparatively, the term “output device,” as described herein, may include any type of output device including, but not limited to, a computer monitor, printer, facsimile machine, or other output devices performing a similar function.

It should be noted that although the diagrams herein may show a specific order and composition of method steps, it is understood that the order of these steps may differ from what is depicted. In a non-limiting example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative implementations. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps, and decision steps.

While this specification contains many specific implementation details, these should not be construed as limitations on the scope of any inventions or of what may be claimed, but rather as descriptions of features specific to particular implementations of the systems and methods described herein. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Having now described some illustrative implementations and implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements, and features discussed only in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The foregoing description of implementations has been presented for purposes of illustration and description. It is not intended to be exhaustive or to limit the disclosure to the precise form disclosed, and modifications and variations are possible in light of the above teachings or may be acquired from this disclosure. The implementations were chosen and described in order to explain the principals of the disclosure and its practical application to enable one skilled in the art to utilize the various implementations and with various modifications as are suited to the particular use contemplated. Other substitutions, modifications, changes, and omissions may be made in the design, operating conditions and implementation of the implementations without departing from the scope of the present disclosure as expressed in the appended claims.

What is claimed is:

1. A low-field multi-directional diffusion-weighted magnetic resonance imaging method, the method comprising:
quantifying motion based on signal variation; and
generating one or more output images based on multi-directional image reconstructions that exclude data based on the quantified motion.

2. The method of claim 1, further comprising acquiring imaging data.

3. The method of claim 2, wherein the imaging data is acquired using an MRI system with a field strength no greater than 0.2 T.

4. The method of claim 3, wherein the MRI system is a portable MRI system that is cartable between locations using one or more battery-powered motors coupled to a set of wheels.

5. The method of claim 2, wherein acquiring imaging data comprises performing a pseudo-trace acquisition encoded along a plurality of axes.

6. The method of claim 1, wherein the multi-directional image reconstructions correspond to a first direction and a second direction that is different from the first direction.

7. The method of claim 6, wherein the first direction is orthogonal to the second direction.

8. The method of claim 1, wherein the multi-directional image reconstructions correspond to a plurality of directions.

9. The method of claim 1, further comprising aligning navigator signal across all segments.

10. The method of claim 1, further comprising generating a reference pattern from an aligned navigator signal for motion level quantification.

11. The method of claim 1, further comprising computing normalized projection residual against a reference pattern.

12. The method of claim 1, further comprising determining for each segment, based on a projection having a normalized residual energy level above a chosen threshold, to reject data from the segment.

13. The method of claim 1, wherein the multi-directional image reconstructions comprise independently reconstructed diffusion-weighted images corresponding to respective diffusion-encoding directions.

14. The method of claim 1, wherein generating the one or more output images comprises generating at least one of a pseudo-trace image or an apparent diffusion coefficient map.

15. The method of claim 1, wherein excluding data based on the quantified motion comprises excluding all data from a shot segment or excluding only a portion of data from the shot segment.

16. The method of claim 1, further comprising, based on the quantified motion, causing reacquisition of imaging data corresponding to at least one excluded shot segment.

17. A low-field MRI system for multi-directional diffusion-weighted magnetic resonance imaging, the MRI system comprising one or more processors configured to:
quantify motion based on signal variation; and
generate one or more output images based on multi-directional image reconstructions that exclude data based on the quantified motion.

18. The MRI system of claim 17, wherein the MRI system has a field strength no greater than 0.2 T.

19. The MRI system of claim 17, wherein the MRI system is cartable between locations using one or more battery-powered motors coupled to a set of wheels.

20. A non-transitory computer-readable medium storing instructions that, when executed by one or more processors of a magnetic resonance imaging system, a computing device, or a computing system in communication with the magnetic resonance imaging system, cause the one or more processors to:
quantify motion based on signal variation; and
generate one or more output images based on multi-directional image reconstructions that exclude data based on the quantified motion.
